(19) United States
(12) Patent Application Publication
(10) Pub. No.: US 2024/0087703 A1
(43) Pub. Date: Mar. 14, 2024

(54) SYSTEM AND METHOD FOR PROVIDING REAL-TIME PRESCRIPTION SHARING AND DISPENSING INFORMATION MONITORING SERVICE BASED ON BLOCKCHAIN NETWORK

(71) Applicant: Electronics and Telecommunications Research Institute, Daejeon (KR)

(72) Inventors: Bumhwi KIM, Daejeon (KR); Kyu Hyung KIM, Daejeon (KR)

(73) Assignee: Electronics and Telecommunications Research Institute, Daejeon (KR)

(21) Appl. No.: 18/101,372

(22) Filed: Jan. 25, 2023

(30) Foreign Application Priority Data
Sep. 13, 2022 (KR) 10-2022-0114819

(51) Int. Cl. G16H 20/10; G16H 80/00

(52) U.S. Cl. CPC G16H 20/10; G16H 80/00

(57) ABSTRACT

Provided is a method for providing a real-time prescription sharing and dispensing information monitoring service based on a blockchain network. The method includes receiving prescription information that is input by a doctor device and that corresponds to medical treatment of the doctor, encrypting and storing the prescription information over a blockchain network, receiving dispensing information that is input by a druggist device and that corresponds to the prescription information, encrypting and storing the dispensing information over the blockchain network, and allowing at least one of the doctor device, the druggist device, and a patient device to selectively inquire about the encrypted prescription information and the encrypted dispensing information according to a predetermined condition.

### CROSS-REFERENCE TO RELATED APPLICATION

This application claims priority to and the benefit of Korean Patent Application No. 10-2022-0114819, filed on Sep. 13, 2022, the disclosure of which is incorporated herein by reference in its entirety.

### BACKGROUND

### 1. Technical Field

The present disclosure relates to a system and method for providing a real-time prescription sharing and dispensing information monitoring service based on a blockchain network.

### 2. Related Art

Today, after a subject that has been quarantined due to COVID-19 is subjected to non-face-to-face treatment, in the non-face-to-face treatment service the subject submits a prescription issued by a doctor to a nearby drugstore and receives a dispensed drug through a delivery service in a non-face-to-face way, or the subject visits a nearby drugstore to which the issued prescription has been delivered and directly receives the dispensed drug. However, in such a situation, a concern about the forgery and alteration of an issued prescription may occur, and the misuse and abuse of drugs may occur by abusing such a non-face-to-face treatment service.

As medicines continue to fall short due to a sudden increase of confirmed cases attributable to COVID-19, the needs for drugstores for alternative dispensing are increased. However, in a current pharmaceutical affairs law, in the case of the same ingredient dispensing, if a druggist wants to dispense medicine and medical supplies having the same components, content, and a dosage form as medicine and medical supplies described in a prescription issued by a doctor by replacing the medicine and medical supplies described in the prescription, the druggist needs to receive the consent of the doctor who has issued the prescription in advance.

Furthermore, the range of the same ingredient dispensing capable of post-notification without prior consent has been specified. Accordingly, a date for post-notification is 3 days without prior consent, and a druggist dispenses medicine and medical supplies based on the date. However, doctors or patients are exposed to a danger of the occurrence of a drug dispensing accident because the doctors or patients distrust alternative dispensing of druggists or distrust the effectiveness of generic drugs.

Another problem is that a doctor and a druggist may gain an advantage by prescribing and dispensing a drug of a specific drug manufacturer.

In order to prevent such a problem, it is necessary for a patient to share and receive a prescription by using his or her device, visit another drugstore, confirm the prescription, and receive a dispensed drug. Conclusionally, there is a need for a technology for sharing and confirming a prescription and dispensing information in real time according to rights.

### SUMMARY

Various embodiments are directed to providing a system and method for providing a real-time prescription sharing and dispensing information monitoring service based on a blockchain network, which can provide a transparent and reliable prescription-dispensing process by encrypting prescription information and dispensing information and enabling the prescription information and dispensing information to be shared based on a blockchain network and allow prescription and dispensing information to be shared and received regardless of a place through the sharing characteristic of a blockchain network.

However, an object to be solved by the present disclosure is not limited to the aforementioned object, and other objects may be present.

According to a first aspect of the present disclosure, a method of providing a real-time prescription sharing and dispensing information monitoring service based on a blockchain network includes receiving prescription information that is input by a doctor device and that corresponds to medical treatment of the doctor, encrypting and storing the prescription information over a blockchain network, receiving dispensing information that is input by a druggist device and that corresponds to the prescription information, encrypting and storing the dispensing information over the blockchain network, and allowing at least one of the doctor device, the druggist device, and a patient device to selectively inquire about the encrypted prescription information and the encrypted dispensing information according to a predetermined condition.

Furthermore, according to a second aspect of the present disclosure, a system for providing a real-time prescription sharing and dispensing information monitoring service based on a blockchain network includes a communication unit configured to transmit and receive data to and from a doctor device, a druggist device, and a patient device, memory in which a program for real-time prescription sharing and dispensing information monitoring using an encryption method based on a blockchain network has been stored, and a processor configured to execute the program stored in the memory. In this case, the processor encrypts and stores prescription information that is input by the doctor device and that corresponds to medical treatment of the doctor and dispensing information that is input by the druggist device and that corresponds to the prescription information over the blockchain network, and allows the prescription information and the dispensing information to be selectively inquired by at least one of the doctor device, the druggist device and the patient device according to a predetermined condition, by executing the program.

A computer program according to another aspect of the present disclosure executes the method of providing a real-time prescription sharing and dispensing information monitoring service based on a blockchain network and is stored in a computer-readable recording medium.

Other details of the present disclosure are included in the detailed description and the drawings.

According to an embodiment of the present disclosure, as described above, underlying disease information, medication information, prescription information, and dispensing information corresponding to personal information of a patient can be encrypted based on a blockchain network and can be shared and inquired according to a predetermined condition. Accordingly, corresponding information cannot be exposed and not forged and altered, and can also be monitored in real time.

The effects of the present disclosure are not limited to the aforementioned effects, and other effects which are not mentioned herein will be clearly understood by those skilled in the art from the following descriptions.

### BRIEF DESCRIPTION OF THE DRAWINGS

FIG. 1 is a diagram for describing a service provision system according to an embodiment of the present disclosure.

FIG. 2 is a diagram illustrating a structure of the entire system according to an embodiment of the present disclosure.

FIGS. 3A to 3C are flowcharts of a service provision method according to an embodiment of the present disclosure.

### DETAILED DESCRIPTION

Advantages and characteristics of the present disclosure and a method for achieving the advantages and characteristics will become apparent from the embodiments described in detail later in conjunction with the accompanying drawings. However, the present disclosure is not limited to the disclosed embodiments, but may be implemented in various different forms. The embodiments are merely provided to complete the present disclosure and to fully notify a person having ordinary knowledge in the art to which the present disclosure pertains of the category of the present disclosure. The present disclosure is merely defined by the category of the claims.

Terms used in this specification are used to describe embodiments and are not intended to limit the present disclosure. In this specification, an expression of the singular number includes an expression of the plural number unless clearly defined otherwise in the context. The term “comprises” and/or “comprising” used in this specification does not exclude the presence or addition of one or more other elements in addition to a mentioned element. Throughout the specification, the same reference numerals denote the same elements. The term “and/or” includes each of mentioned elements and all combinations of one or more of mentioned elements. Although the terms “first”, “second”, etc. are used to describe various components, these elements are not limited by these terms. These terms are merely used to distinguish between one element and another element. Accordingly, a first element mentioned hereinafter may be a second element within the technical spirit of the present disclosure.

All terms (including technical and scientific terms) used in this specification, unless defined otherwise, will be used as meanings which may be understood in common by a person having ordinary knowledge in the art to which the present disclosure pertains. Furthermore, terms defined in commonly used dictionaries are not construed as being ideal or excessively formal unless specially defined otherwise.

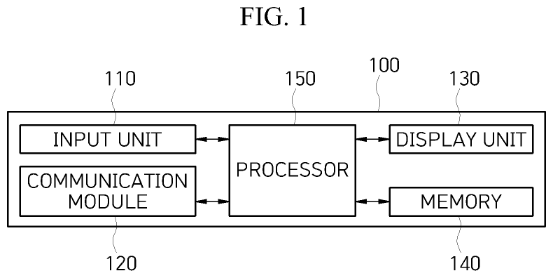

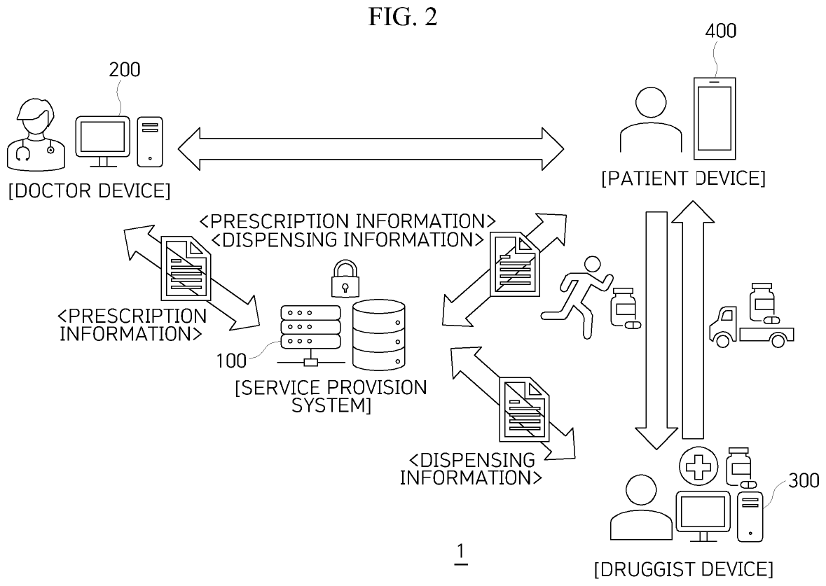

Hereinafter, a system 100 for providing a real-time prescription sharing and dispensing information monitoring service based on a blockchain network (hereinafter referred to as a “service provision system 100”) according to an embodiment of the present disclosure is described with reference to FIGS. 1 and 2.

FIG. 1 is a diagram for describing a service provision system according to an embodiment of the present disclosure.

The service provision system 100 according to an embodiment of the present disclosure includes an input unit 110, a communication unit 120, a display unit 130, memory 140, and a processor 150.

The input unit 110 generates input data in response to a user input to the service provision system 100. The user input may include a user input relating to data to be processed by the service provision system 100.

The input unit 110 includes at least one input means. The input unit 110 may include a keyboard, a key pad, a dome switch, a touch panel, a touch key, a mouse, and a menu button.

The communication unit 120 transmits and receives data between the internal components or performs communication with an external device, such as an external server. In an embodiment, the communication unit 120 may transmit and receive data to and from a doctor device, a druggist device, and a patient device. The communication unit 120 may include both a wired communication unit and a wireless communication unit. The wired communication unit may be implemented by using a power line communication device, a telephone line communication device, cable home (MoCA), Ethernet, IEEE1294, an integrated wired home network, or an RS-485 controller. Furthermore, the wireless communication unit may be constituted with a module for implementing a function, such as a wireless LAN (WLAN), Bluetooth, a HDR WPAN, UWB, RoLa, ZigBee, Impulse Radio, 60 GHz WPAN, Binary-CDMA, a wireless USB technology, a wireless HDMI technology, 5^{th} generation (5G) communication, long term evolution-advanced (LTE-A), long term evolution (LTE), or wireless fidelity (Wi-Fi).

The display unit 130 displays display data according to an operation of the service provision system 100. The display unit 130 includes a liquid crystal display (LCD), a light emitting diode (LED) display, an organic LED (OLED) display, a micro electro mechanical systems (MEMS) display, and an electronic paper display. The display unit 130 may be implemented as a touch screen by being coupled with the input unit 110.

A program for real-time prescription sharing and dispensing information monitoring using an encryption method based on a blockchain network is stored in the memory 140. In this case, the memory 140 collectively refers to nonvolatile storage and volatile storage that continue to retain information stored therein although power is not supplied thereto. For example, the memory 140 may include NAND flash memory, such as a compact flash (CF) card, a secure digital (SD) card, a memory stick, a solid-state drive (SSD), and a micro SD card, magnetic computer storage, such as a hard disk drive (HDD), and optical disc drives, such as CD-ROM and DVD-ROM.

The processor 150 may control at least one other component (e.g., a hardware or software component) of the service provision system 100 by executing software, such as a program, and may perform various data processing or operations.

FIG. 2 is a diagram illustrating a structure of the entire system 1 according to an embodiment of the present disclosure.

In an embodiment of the present disclosure, the entire system 1 includes the service provision system 100, a doctor device 200, a druggist device 300, and a patient device 400.

In an embodiment of the present disclosure, after using the non-face-to-face treatment service, a patient who has contracted COVID-19 having strong infectivity and must be quarantined may share a prescription issued by a doctor through a drugstore near the patient or through his or her patient device over a blockchain network, may visit a desired drugstore, and may receive a dispensed drug.

Simultaneously, whether the dispensed drug is identical with a drug dispensed by the doctor may also be shared by the doctor. Accordingly, the patient can receive the dispensed drug with more peace of mind.

A patient may receive a dispensed drug through delivery without visiting a drugstore or may directly visit the drugstore and receive the prescribed drug.

In the first case, a prescription issued by a doctor is stored in the service provision system 100. When the patient designates a drugstore by which the prescription is to be shared, the service provision system 100 shares the prescription with the designated drugstore. Thereafter, the patient may receive the dispensed drug from the designated drugstore through delivery.

In the second case, the prescription issued by the doctor is stored in the service provision system 100. When the patient designates a drugstore by which the prescription is to be shared, the service provision system 100 shares the prescription with the designated drugstore. Thereafter, the patient may visit the designated drugstore and directly receive the prescribed drug.

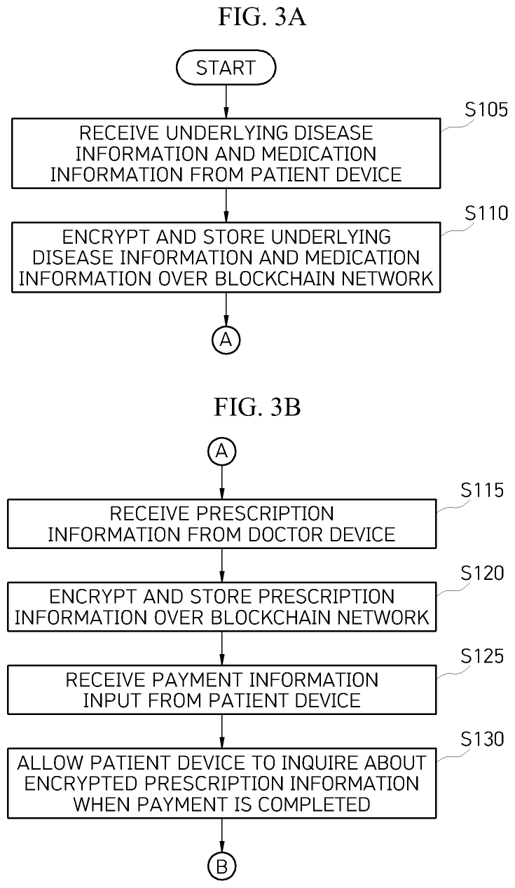

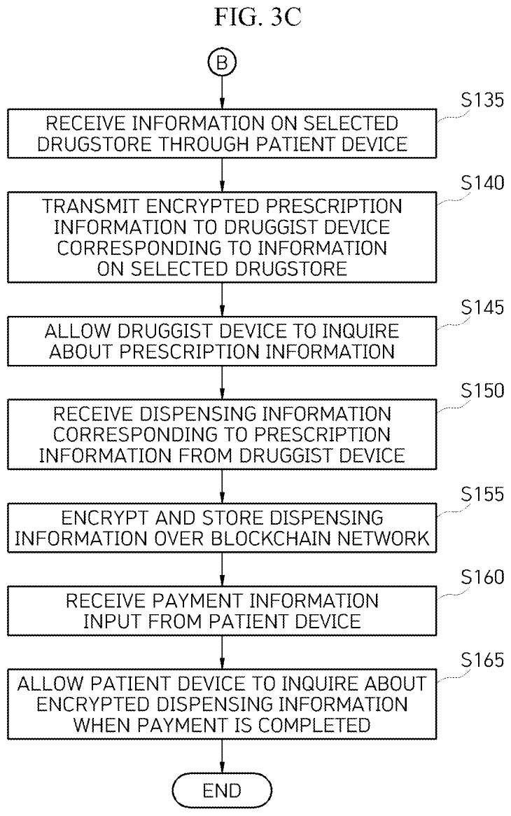

Hereinafter, a method of providing a real-time prescription sharing and dispensing information monitoring service based on a blockchain network (hereinafter referred to as a “service provision method”), which is performed by the service provision system 100, is described with reference to FIGS. 3A to 3C.

FIGS. 3A to 3C are flowcharts of a service provision method according to an embodiment of the present disclosure.

First, when a patient inputs his or her underlying disease information and medication information to the patient device 400 in order to use the real-time prescription sharing and dispensing information monitoring service, the service provision system 100 receives the underlying disease information and medication information from the patient device 400 (S105).

Thereafter, the service provision system 100 encrypts and stores the underlying disease information and medication information over a blockchain network (S110).

Thereafter, when a doctor treats the patient in a non-face-to-face way, the doctor inputs prescription information corresponding to the medical treatment through the doctor device 200. The service provision system 100 receives the prescription information from the doctor device 200 (S115). Furthermore, the service provision system 100 encrypts and stores the received prescription information over the blockchain network (S120).

As an embodiment, before encrypting and storing the prescription information, the service provision system 100 may check whether side effect information is detected through the drug utilization review (DUR) into the prescription information issued by the doctor, based on underlying disease information and medication information of the patient which have been previously stored.

If the side effect information is detected as a result of the check, the service provision system 100 may transmit the detected side effect information to the doctor device 200, and may check whether side effect information for new prescription information is detected through the DUR into the new prescription information when receiving the new prescription information from the doctor device 200. Furthermore, if side effect information is not detected as a result of the re-check, the service provision system 100 may encrypt and store the new prescription information.

In contrast, if side effect information is not detected as a result of the check, the service provision system 100 may encrypt and store the prescription information. That is, if there is no problem with the prescription, the prescription information is recorded normally. The service provision system 100 receives a payment information input according to the medical treatment from the patient device 400 (S125), and may allow the patient device 400 to inquire about the encrypted prescription information when payment corresponding to the payment information input is completed normally (S130).

Next, when the patient selects a drugstore to which the prescription information is to be transmitted, the service provision system 100 receives information on the selected drugstore through the patient device 400 (S135). Furthermore, the service provision system 100 transmits the encrypted prescription information to the druggist device 300 corresponding to the received information on the drugstore (S140). Thereafter, when checking that the druggist device 300 has received the prescription information or when transmitting the prescription information, the service provision system 100 allows the prescription information to be inquired (S145).

In an embodiment of the present disclosure, a patient may select a drugstore to which prescription information is to be transmitted by using two methods.

As an embodiment, the service provision system 100 may provide the patient device 400 with information on at least one drugstore within a certain radius based on information on the location of the patient device 400, and may receive, through the patient device 400, information on any one drugstore selected by the patient among the provided information on the drugstore.

As another embodiment, the service provision system 100 may provide the patient device 400 with information on at least one drugstore that is included in information on an area that has been selected by the patient as an area at which the patient will directly receive a dispensed drug, and may receive, through the patient device 400, information on any one drugstore that has been selected by the patient, among the pieces of provided information on the drugstore.

The service provision system 100 may allow the prescription information to be transmitted to and inquired by the druggist device 300 corresponding to the provided information on the drugstore that has been selected as described above.

After the druggist device 300 receives the prescription information, as a dispensing behavior is completed, the service provision system 100 receives dispensing information that is input by the druggist device 300 and that corresponds to the prescription information (S150), and encrypts and stores the dispensing information over the blockchain network (S155).

Thereafter, the service provision system 100 receives, from the patient device 400, a payment information input relating to a dispensed drug cost corresponding to the dispensing information (S160), and may allow the patient device 400 to inquire about the encrypted dispensing information when payment corresponding to the payment information input is completed normally (S165).

Furthermore, when the dispensing information is stored, the service provision system 100 may transmit the encrypted dispensing information to the doctor device 200 that has issued the corresponding prescription information. The doctor device 200 that has received the encrypted dispensing information may confirm whether a drug has been dispensed as prescribed. Accordingly, a danger attributable to a drug misuse before a patient receives a dispensed drug can be prevented.

As described above, according to the embodiments of the present disclosure, underlying disease information, medication information, prescription information, and dispensing information corresponding to personal information of a patient can be encrypted based on a blockchain network and can be shared and inquired according to a predetermined condition. Accordingly, corresponding information cannot be exposed and not forged and altered, and can also be monitored in real time.

In the aforementioned description, steps S110 to S230 may be further divided into additional steps or may be combined into smaller steps depending on an implementation example of the present disclosure. Furthermore, some steps may be omitted, if necessary, and the sequence of steps may be changed. Furthermore, although contents are omitted, the contents described with reference to FIGS. 1 and 2 and the contents described with reference to FIGS. 3A to 3C may be mutually applied.

The method of providing a real-time prescription sharing and dispensing information monitoring service based on a blockchain network according to an embodiment of the present disclosure may be implemented in the form of a program (or application) in order to be executed by being combined with a computer, that is, hardware, and may be stored in a medium.

The aforementioned program may include a code coded in a computer language, such as C, C++, JAVA, Ruby, or a machine language which is readable by a processor (CPU) of a computer through a device interface of the computer in order for the computer to read the program and execute the methods implemented as the program. Such a code may include a functional code related to a function, etc. that defines functions necessary to execute the methods, and may include an execution procedure-related control code necessary for the processor of the computer to execute the functions according to a given procedure. Furthermore, such a code may further include a memory reference-related code indicating at which location (address number) of the memory inside or outside the computer additional information or media necessary for the processor of the computer to execute the functions needs to be referred. Furthermore, if the processor of the computer requires communication with any other remote computer or server in order to execute the functions, the code may further include a communication-related code indicating how the processor communicates with the any other remote computer or server by using a communication unit of the computer and which information or media needs to be transmitted and received upon communication.

The stored medium means a medium, which semi-permanently stores data and is readable by a device, not a medium storing data for a short moment like a register, a cache, or memory. Specifically, examples of the stored medium include ROM, RAM, CD-ROM, a magnetic tape, a floppy disk, optical data storage, etc., but the present disclosure is not limited thereto. That is, the program may be stored in various recording media in various servers which may be accessed by a computer or various recording media in a computer of a user. Furthermore, the medium may be distributed to computer systems connected over a network, and a code readable by a computer in a distributed way may be stored in the medium.

The description of the present disclosure is illustrative, and a person having ordinary knowledge in the art to which the present disclosure pertains will understand that the present disclosure may be easily modified in other detailed forms without changing the technical spirit or essential characteristic of the present disclosure. Accordingly, it should be construed that the aforementioned embodiments are only illustrative in all aspects, and are not limitative. For example, elements described in the singular form may be carried out in a distributed form. Likewise, elements described in a distributed form may also be carried out in a combined form.

The scope of the present disclosure is defined by the appended claims rather than by the detailed description, and all changes or modifications derived from the meanings and scope of the claims and equivalents thereto should be interpreted as being included in the scope of the present disclosure.

What is claimed is:

1. A method of providing a real-time prescription sharing and dispensing information monitoring service based on a blockchain network, the method being performed by a computer and comprising:
receiving prescription information that is input by a doctor device and that corresponds to medical treatment of the doctor;
encrypting and storing the prescription information over a blockchain network;
receiving dispensing information that is input by a druggist device and that corresponds to the prescription information;
encrypting and storing the dispensing information over the blockchain network; and
allowing at least one of the doctor device, the druggist device, and a patient device to selectively inquire about the encrypted prescription information and the encrypted dispensing information according to a predetermined condition.

2. The method of claim 1, further comprising receiving a payment information input according to the medical treatment from the patient device,
wherein the allowing of the encrypted prescription information and the encrypted dispensing information to be selectively inquired comprises allowing the patient device to inquire about the encrypted prescription information when payment corresponding to the payment information input is completed.

3. The method of claim 1, further comprising:
receiving, through the patient device, information on a drugstore to which the prescription selected and input by a patient is to be transmitted; and
transmitting the encrypted prescription information to a druggist device corresponding to the received information on the drugstore,
wherein the allowing of the encrypted prescription information and the encrypted dispensing information to be selectively inquired comprises allowing the druggist device to inquire about the prescription information when receiving the prescription information.

4. The method of claim 3, wherein the receiving of, through the patient device, the provided information on the drugstore to which the prescription selected and input by the patient is to be transmitted comprises:
providing the patient device with information on at least one drugstore within a certain radius based on information on a location of the patient device; and
receiving, through the patient device, information on any one drugstore selected by the patient, among the provided information on the drugstore.

5. The method of claim 3, wherein the receiving of, through the patient device, the provided information on the drugstore to which the prescription selected and input by the patient is to be transmitted comprises:
providing the patient device with information on at least one drugstore, which is included in information on an area selected by the patient; and
receiving, through the patient device, information on any one drugstore selected by the patient, among the provided information on the drugstore.

6. The method of claim 1, further comprising receiving, from the patient device, a payment information input relating to a dispensed drug cost corresponding to the dispensing information,
wherein the allowing of the encrypted prescription information and the encrypted dispensing information to be selectively inquired comprises allowing the patient device to inquire about the encrypted dispensing information when payment corresponding to the payment information input is completed.

7. The method of claim 1, further comprising:
receiving underlying disease information and medication information from the patient device; and
encrypting and storing the underlying disease information and the medication information over the blockchain network.

8. The method of claim 7, wherein the encrypting and storing of the prescription information over the blockchain network comprises:
checking whether side effect information for the prescription information is detected through a drug utilization review (DUR) into the prescription information based on the stored underlying disease information and medication information; and
storing the prescription information when side effect information is not detected as a result of the check.

9. The method of claim 8, wherein the encrypting and storing of the prescription information over the blockchain network comprises:
transmitting the side effect information to the doctor device when the side effect information is detected as a result of the check;
checking whether side effect information for new prescription information is detected through the DUR into the new prescription information when the new prescription information is received from the doctor device; and
storing the new prescription information when side effect information is not detected as a result of the check.

10. A system for providing a real-time prescription sharing and dispensing information monitoring service based on a blockchain network, the system comprising:
a communication unit configured to transmit and receive data to and from a doctor device, a druggist device, and a patient device;
memory in which a program for real-time prescription sharing and dispensing information monitoring using an encryption method based on a blockchain network has been stored; and
a processor configured to execute the program stored in the memory,
wherein the processor, by executing the program,
encrypts and stores prescription information that is input by the doctor device and that corresponds to medical treatment of the doctor and dispensing information that is input by the druggist device and that corresponds to the prescription information over the blockchain network, and
allows the prescription information and the dispensing information to be selectively inquired by at least one of the doctor device, the druggist device and the patient device according to a predetermined condition.

11. The system of claim 10, wherein the processor
receives, from the patient device, a payment information input according to the medical treatment or a payment information input relating to a dispensed drug cost corresponding to the dispensing information through the communication unit, and
allows the patient device to inquire about the encrypted prescription information or the encrypted dispensing information when payment through the patient device is completed.

12. The system of claim 10, wherein when receiving, from the patient device, information on a drugstore to which the prescription selected and input by the patient is to be transmitted through the communication unit, the processor allows the druggist device corresponding to the received information on the drugstore to inquire about the encrypted prescription information, and transmits the encrypted prescription information to the druggist device.

13. The system of claim 12, wherein the processor
provides the patient device with information on at least one drugstore within a certain radius based on information on a location of the patient device or at least one drugstore included in information on an area selected by the patient, and
transmits the prescription information to the druggist device corresponding to information on any one drugstore selected by the patient, among the provided information on the drugstore, when receiving the information on the any one drugstore through the patient device.

14. The system of claim 10, wherein the processor
encrypts and stores underlying disease information and medication information over the blockchain network when receiving the underlying disease information and the medication information from the patient device,
checks whether side effect information for the prescription information is detected through a drug utilization review (DUR) into the prescription information, and
stores the prescription information when side effect information is not detected as a result of the check.
